(19) United States
(12) Patent Application Publication
(10) Pub. No.: US 2024/0350381 A1
(43) Pub. Date: Oct. 24, 2024

(54) FRAGRANCES WITH IMPROVED LONG-LASTING PERFORMANCE

(71) Applicant: Firmenich SA, Satigny (CH)

(72) Inventors: Aude DAUGERON JOUAULT, Neuilly-sur-Seine (FR); Arnaud STRUILLOU, Satigny (CH); Addi FADEL, Plainsboro, NJ (US); Madelyne PHAM, Neuilly-sur-Seine (FR); Artem KIRSHON, Plainsboro, NJ (US)

(73) Assignee: Firmenich SA, Satigny (CH)

(21) Appl. No.: 18/685,614

(22) PCT Filed: Sep. 9, 2022
(86) PCT No.: PCT/EP2022/075075

§ 371 Date: Feb. 22, 2024

Related U.S. Application Data
(60) Provisional application No. 63/242,366, filed on Sep. 9, 2021.
(60) Provisional application No. 63/253,723, filed on Oct. 8, 2021.

(30) Foreign Application Priority Data
Oct. 11, 2021 (EP) 21201805.5

(51) Int. Cl. A61K 8/34; A61K 8/39; A61Q 13/00

(52) U.S. Cl. CPC A61K 8/34; A61K 8/39; A61Q 13/00

(57) ABSTRACT

The present invention relates to fragrances, consumer products, and uses thereof having long-lasting fragrance performance based upon at least one fixative selected based upon Hansen Solubility Parameters and perfume raw materials having moderate and/or high volatility.

### CROSS REFERENCE TO RELATED APPLICATIONS

The present application claims the benefit of priority of European Application No. 21201805.5, filed Oct. 11, 2021, U.S. Provisional Application No. 63/253,723, filed on Oct. 8, 2021, and U.S. Provisional Application No. 63/242,366, filed on Sep. 9, 2021. The entire contents of these applications are explicitly incorporated herein by this reference.

### FIELD OF THE INVENTION

The present invention relates to the field of fragrances. More particularly, the present invention relates to fragrances having improved long-lasting performance.

### BACKGROUND

Long-lasting performance has long been sought after in the fragrance industry. Fragrance long-lastingness and tenacity is a key element of fragrance performance and is a desired consumer benefit in applications such as fine fragrance and anti-perspirant/deodorants. This characteristic has generally been pursued using a pyramid fragrance construction, which includes selecting a large quantity of perfumery ingredients with low volatility (base notes), an intermediate quantity of perfumery ingredients with moderate volatility (middle notes), and the lowest quantity of perfumery ingredients with high volatility (top notes).

### SUMMARY OF THE INVENTION

The present invention uniquely combines fixative selection based upon Hansen Solubility Parameters (HSP) with a perfume ingredient having a middle and/or top note to identify superior performing fixatives, which have improved fragrance long-lastingness, linearity and freshness sustainability.

The present invention encompasses the selection of fragrance fixatives based on HSP values for leave-on products such as, for example, eau de toilette, eau de parfum, body sprays, deodorant, anti-perspirant and air care products.

A fragrance according to the present invention may comprise:
(a) at least one perfume ingredient selected from the group consisting of: a perfume ingredient having a top note, a perfume ingredient having a middle note, and a combination thereof; and
(b) at least one fixative having:
   i. at least two Hansen Solubility Parameters selected from a first group consisting of: an atomic dispersion force (δ_{d}) from 12 to 20, a dipole moment (δₚ) from 1 to 7, and a hydrogen bonding (δₕ) from 2.5 to 11, when in solution with a compound having a vapor pressure greater than 0.08 Torr at 22° C.; and
   ii. at least two Hansen Solubility Parameters selected from a second group consisting of: an atomic dispersion force (δ_{d}) from 14 to 20, a dipole moment (δₚ) from 1 to 8, and a hydrogen bonding (δₕ) from 4 to 11, when in solution with a compound having a vapor pressure range of 0.0008 to 0.08 Torr at 22° C.

The fragrance may further comprise an alcohol and water.

In an aspect of the present invention, group (i) may be selected from the group consisting of: an atomic dispersion force (δ_{d}) of 15.84±3.56, a dipole moment (δₚ) of 4.15±2.65, and a hydrogen bonding (δₕ) of 6.72±4.11; and group (ii) is selected from the group consisting of: an atomic dispersion force (δ_{d}) of 16.86±2.72, a dipole moment (δₚ) of 4.61±3.10, and a hydrogen bonding (δₕ) of 7.66±3.29.

A fixative according to the present invention may be, for example, octan-1-ol, octan-2-ol, 2-butyloctan-1-ol, 11-methyldodecan-1-ol, 2-hexyldecan-1-ol, 14-methylpentadecan-1-ol, 16-methylheptadecan-1-ol, 2-octyldecan-1-ol, 2-octyldodecan-1-ol, 2-decyltetradecan-1-ol, 2-dodecylhexadecan-1-ol, 2-tetradecyloctadecan-1-ol, [3-(2-ethylhexanoyloxy)-2,2-dimethylpropyl] 2-ethylhexanoate, 3-tetradecoxypropan-1-ol, or a combination thereof.

A fixative according to the present invention may have little odor or be non-odiferous.

In an aspect of the present invention, the fixative comprises 0.1% or greater by weight of the fragrance.

In a further aspect, the fixative is in a ratio of at least 1/10 to the at least one perfume ingredient. The fixative may also be in a ratio of at least 1/4 compared to the at least one perfume ingredient.

In aspects of the present invention, the fixative is represented by the formula (I):

CH₃-(CH₂)ₓ-CHZ-(CH₂)_{y}-(CH₃) (I)

wherein Z is a CH₂OH, a CHO, a CO₂H, a CH₂NH₂ or a CH₂SH group;
x is an integer between 3 and 15;
y is an integer between 3 and 15; and provided that |x-y| is less than 8.

A fixative according to the present invention may be a profragrance.

The present invention encompasses consumer products comprising a fragrance of the present invention.

### BRIEF DESCRIPTION OF THE FIGURES

FIG. 1 shows an illustration of the Hansen Space defined in the 3 directions by each interaction (D, P and H) and with R0 being the radius of the sphere of solubility characteristic of the solute.

FIG. 2 shows the total area sums at 1 hour evaporation of different levels of PPG-20 Methyl Glucose Ether (EDT B) vs. different levels of Isocetyl Alcohol (EDT C).

FIG. 3 shows the total area sums at 2 hours evaporation of different levels of PPG-20 Methyl Glucose Ether (EDT B) vs. different levels of Isocetyl Alcohol (EDT C).

FIG. 4 shows the total area sums at 4 hours evaporation of different levels of PPG-20 Methyl Glucose Ether (EDT B) vs. different levels of Isocetyl Alcohol (EDT C).

FIG. 5 shows direct injection data for each individual compound at 2 hours evaporation of RAHTI alone (EDT A) vs. 5% PPG-20 Methyl Glucose Ether with RAHTI (EDT B) vs. 5% Isocetyl Alcohol with RAHTI (EDT C).

FIG. 6 shows the fragrance area sums at 4 hours evaporation of EDT D (Profragrance 1), EDT E (Profragrance 2) and EDT F (Profragrance 3) vs. control EDT A.

FIG. 7 shows the total area sums throughout evaporation of RAHT2 alone (EDT1 RAHT2) vs. 5% Hexyldecanol with RAHT2 (EDT2 RAHT2) vs. 5% Octyldodecanol with RAHT2 (EDT3 RAHT2).

FIG. 8 shows direct injection data for each individual compound at 2 hours evaporation of RAHT2 alone (EDT1 RAHT2) vs. 5% Hexyldecanol with RAHT2 (EDT2 RAHT2) vs. 5% Octyldodecanol with RAHT2 (EDT3 RAHT2).

FIG. 9 shows direct injection data for each individual compound at 4 hours evaporation of RAHT2 alone (EDT1 RAHT2) vs. 5% Hexyldecanol with RAHT2 (EDT2 RAHT2) vs. 5% Octyldodecanol with RAHT2 (EDT3 RAHT2).

FIG. 10 shows direct injection data for each individual compound at 6 hours evaporation of RAHT2 alone (EDT1 RAHT2) vs. 5% Hexyldecanol with RAHT2 (EDT2 RAHT2) vs. 5% Octyldodecanol with RAHT2 (EDT3 RAHT2).

FIG. 11 shows that a fragrance with hexyldecanol (Jarcol I-16 N) was perceived more intense after 2 hours and 4 hours dry down compared to fragrance with isocetyl alcohol (ICA).

### DETAILED DESCRIPTION

A “fixative” or “modulator” according to the present invention is a material that modulates the vapor pressure of a perfumery ingredient, delaying the perfumery ingredient's evaporation profile. A fixative or modulator may be non-odoriferous or a profragrance with fixative properties.

A “properfume” or “profragrance” is a compound that is able to release one, two or more perfume ingredients, also termed PRMs (perfumery raw materials), upon external influence in a way that prolongs the perfuming effect of the PRMs. In the present invention, the terms “properfume” or “profragrance” are used interchangeably. The perfumery raw materials may be released from the pro-perfume compound by one or more mechanisms. For example, the perfumery raw materials may be released from the pro-perfume compound by (chemical) cleavage of the pro-perfume compound. The external influence leading to the cleavage of the pro-perfume compound may be light. By “light”, any form of electromagnetic radiation is meant, which is not limited to any particular wavelength. The release of PRMs from such a pro-perfume compound is usually more effective at lower wavelengths (higher energy input). The cleavage of a certain pro-perfume compound may also be triggered by air/oxygen. Thereby, the PRMs may be released from the pro-perfume compound by oxidation in the presence of air (ambient air) or oxygen. Moreover, the PRMs may be released from a certain pro-perfume compound by heat. By “heat”, it is meant any energy input that is caused by increased temperature. Further, the PRMs may be released from a certain properfume compounds by moisture. Such a properfume compound may show chemical bonds that are susceptible to water-induced cleavage and may thus be cleaved in the presence of water. In some cases, a certain pH-value may induce and/or support the cleavage. Further, the PRMs may be released from a certain pro-perfume compound upon exposure to enzymes. Such a pro-perfume compound may show chemical bonds that can efficiently be cleaved in the presence of enzymes. In some cases, the PRMs may be released from a certain properfume compound not only based on one type of release mechanism but based on two or more of the above-mentioned types simultaneously, such as for example release by air/oxygen and moisture. Typically, the properfume itself has a low volatility, and is ideally (almost) odorless. The properfume may be advantageously characterized by a vapor pressure below 0.01 Pa, as obtained by calculation using the software EPIwin v. 3.10 (2000, available at the US Environmental Protection Agency). According to one embodiment, the vapor pressure is below 0.001 Pa. The properfume may also be advantageously characterized by a molecular weight above 270, even above 300, even above 350. The terms “properfume” or “profragrance” have the normal meaning in the art as for example reported in A. Herrmann, Angew. Chem. Int. Ed., 2007, 46, 5836-5863. The profragrance may be in a form of a alpha-ketoester, alpha-ketoacid, a enolether, a Knoevenagel adduct, a Michael adduct, an ester, an α,β-unsaturated ester, a diester, a siloxane, an imine, a cinnamyl ether, a heterocycle such as an aminal, imidazolidinone or oxazolidine, Non-limiting examples of suitable properfume may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 3-(dodecylsulfonyl)-1-(2,6,6-trimethylcyclohex-3-en-1-yl) butan-1-one, a linear polysiloxane co-polymer of (3-mercaptopropyl) (methyl) dimethoxysilane, 2-(dodecylthio) octan-4-one, 2-(dodecylsulfonyl) octan-4-one, 4-oxooctan-2-yl dodecanoate, 2-phenylethyl oxo (phenyl) acetate, 3,7-dimethylocta-2,6-dien-1-yl oxo (phenyl) acetate, (Z)-hex-3-en-1-yl oxo (phenyl) acetate, 3,7-dimethyl-2,6-octadien-1-yl hexadecanoate, bis (3,7-dimethylocta-2,6-dien-1-yl) succinate, (2E,6Z)-2,6-nonadienyl hexadecanoate, (2E,6Z)-2,6-nonadien-1-yl tetradecanoate, (2E,6Z)-2,6-nonadien-1-yl dodecanoate, (2-((2-methylundec-1-en-1-yl) oxy) ethyl) benzene, 1-methoxy-4-(3-methyl-4-phenethoxybut-3-en-1-yl) benzene, (3-methyl-4-phenethoxybut-3-en-1-yl) benzene, 1-(((Z)-hex-3-en-1-yl) oxy)-2-methylundec-1-ene, (2-((2- methylundec-1-en-1-yl) oxy) ethoxy) benzene, 2-methyl-1-(octan-3-yloxy) undec-1-ene, 1-methoxy-4-(1-phenethoxyprop-1-en-2-yl) benzene, 1-methyl-4-(1-phenethoxyprop-1-en-2-yl) benzene, 2-(1-phenethoxyprop-1-en-2-yl) naphthalene, (2-phenethoxyvinyl) benzene, 2-(1-((3,7-dimethyloct-6-en-1-yl) oxy) prop-1-en-2-yl) naphthalene, (2-((2-pentylcyclopentylidene) methoxy) ethyl) benzene, 4-allyl-2-methoxy-1-((2-methoxy-2-phenylvinyl) oxy) benzene, (2-((2-heptylcyclopentylidene) methoxy) ethyl) benzene, 1-methoxy-4-(1-phenethoxyprop-1-en-2-yl) benzene, (2-((2-methyl-4-(2,6,6-trimethylcyclohex-1-en-1-yl) but-1-en-1-yl) oxy) ethyl) benzene, 1-methoxy-4-(2-methyl-3-phenethoxyallyl) benzene, (2-((2-isopropyl-5-methylcyclohexylidene) methoxy) ethyl) benzene, 1-isopropyl-4-methyl-2-((2-pentylcyclopentylidene) methoxy) benzene, 2-methoxy-1-((2-pentylcyclopentylidene) methoxy)-4-propylbenzene, 2-ethoxy-1-((2-methoxy-2-phenylvinyl) oxy)-4-methylbenzene, 3-methoxy-4-((2-methoxy-2-phenylvinyl) oxy) benzaldehyde, 1-isopropyl-2-((2-methoxy-2-phenylvinyl) oxy)-4-methylbenzene, 4-((2-(hexyloxy)-2-phenylvinyl) oxy)-3-methoxybenzaldehyde or a mixture thereof. Particularly, profragrance with fixative properties may be 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone or (2-((2-methylundec-1-en-1-yl) oxy)ethyl)benzene.

A fixative according to the present invention includes materials selected based on solubility parameters which are thought to enable soft interactions with volatile perfume raw materials (PRMs). The odor contributions of the fixatives are low to non-odoriferous to impart minimum olfactive impact on the fragrance mixture in which they are contained.

In aspects of the present invention, a formulation according to the present invention includes:

TABLE 1

| | Wt % Range by Weight of |
|---|---|
| | the Formulation |
| ETOH 200 proof | 30 to 90 |
| Perfume raw material | 0.2 to 50 |
| Fixative | 0.01 to 60 |
| Water | QSP |
| Other hydrophilic solvents | Up to 10% |
| Dyes, stabilizers and actives | — |

In other aspects, a formulation according to the present invention includes:

TABLE 2

| | Wt % Range by Weight of |
|---|---|
| | the Formulation |
| ETOH 200 proof | 50 to 80 |
| Perfume raw material | 1 to 30 |
| Fixative | 0.2 to 20 |
| Water | QSP |
| Other hydrophilic solvents | Up to 10% |
| Dyes, stabilizers and actives | — |

### a. Hansen Solubility Parameters

Fixatives according to the present invention have HSPs optimized for affinity for top and middle notes. HSPs are physicochemical parameters used to estimate the type of interactive forces responsible for compatibility between materials. A full description of HSP and its application to fragrance design is described in WO2020234154, which is hereby incorporated by reference in its entirety.

The basis of the HSP is the assumption that the cohesive energy (E) may be divided into three parts corresponding to atomic dispersion (E_{d}), molecular dipolar interactions (Eₚ), and hydrogen-bonding interactions (Eₕ). Similarly, the total solubility parameter can be divided into three components corresponding to the above-mentioned different types of molecular interactions: dispersion (δ_{d}), polar (δₚ), and hydrogen-bonding (δₕ).

The Hansen solubility parameters are

### 1. Atomic Dispersion Forces δ_{d}

The dispersion term of HSP is regarded as being based on the dispersion energy. Even in systems with no heteroatoms such as oxygen and nitrogen, charge distributions may be created due to movement of electrons. The electric field generated by these charge distributions creates the dispersion attraction between molecules.

This Van der Waals and refractive index based parameter is used to define whether molecules targeted are aliphatic, cycloaliphatic or aromatic and calculated according to method referenced in Blanks and Prausnitz. The dispersion parameter is based on atomic forces and calculated largely using critical temperature Tc which can be in turn estimated using the Lydersen group contributions.

### 2. Polar Solubility Parameter (Dipole Moment) δₚ

The polar solubility parameter is based on the permanent dipole-permanent dipole interactions. It is by equation developed by Hansen and Beerbower:

$${δ}_{p} = 37.4 ( DM ) / {V}^{1 / 2}$$

where DM is the dipole moment of the molecule and V its molar volume.

### 3. Hydrogen Bonding δₕ

Hydrogen-bonding forces can be viewed as the special dipole-dipole interactions that occur between polar molecules where the hydrogen atom attached to an electronegative atom is attracted to another electronegative atom in a different polar molecule such as nitrogen or oxygen. The large difference in electronegativity between hydrogen and the electronegative element renders hydrogen-bonding forces the strongest intermolecular interaction.

The group contribution methods (GSMs), can be used to estimate theoretically solubility parameters. This method is based on the assumption that each functional group of the molecules contributes to the overall thermodynamic property in addition to being additive. As shown in following equations, the three Hansen components can be estimated by Hoftyzer and van Krevelen's method

$${δ}_{d} = \frac{Σ {F}_{d}}{V}$$

$${δ}_{p} = \frac{\sqrt{Σ {F}_{p}^{2}}}{V}$$

$${δ}_{h} = \frac{\sqrt{Σ {E}_{h}}}{V}$$

where F_{d} is the dispersive functional group value, Fₚ is the polar functional group value, Eₕ is the hydrogen-bonding functional group value; V is the molar volume.

The selection of ideal fixatives is based on Hansen's solubility sphere.

Based on the idea of interaction energy additivity, Hansen suggested that the cohesive energy can be represented as a sum of 3 components, corresponding to the 3 different types of interaction: atomic dispersion forces (D), molecular permanent dipole-permanent dipole interactions (P) and electron-exchanging hydrogen bonding interactions (H). He therefore decomposed the Hildebrand solubility parameter in 3 components according to the equation below

$$δ = \sqrt{{δ}_{D}^{2} + {δ}_{P}^{2} + {δ}_{H}^{2}}$$

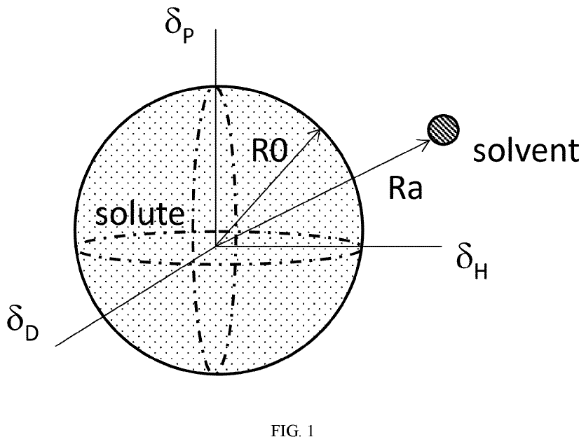

FIG. 1 is an illustration of the Hansen Space defined in the 3 directions by each interaction (D, P and H) and with R0 being the radius of the sphere of solubility characteristic of the solute. Ra represents the distance between the solute solubility parameter (center of the sphere of solubility) and the solvent solubility parameter.

For a fragrance mixture, the simplest way to calculate the HSPs of the mixture is to suppose an ideal mixing of the compounds:
HSPₘᵢₓ=ΣᵢØᵢHSPᵢ, where Øᵢ are the volume fractions of the compounds.

The HSPs can be calculated accurately using the software “HSPiP”. The ratio RED (Relative Energy Difference) is defined as:

$$R E D = \frac{{R}_{a}}{{R}_{0}}$$

The RED number is used as a criterion to gauge the affinity of one compound to another.
If RED<1, the selected fixative has “good” affinity for the other PRM. The affinity limit is achieved when RED=1.
A fixative, for which RED>1 is thought to have no affinity to the said PRM.

The present invention encompasses the use of non-odoriferous fixatives in hydroalcoholic perfume solution. The fixatives are present in a concentration of 0.1% or higher relative to the weight of the perfume solution and have affinity for top and middle notes based on HSP values. The fixatives have a ratio of at least 1/10 or preferably at least 1/4 compared to the fragrance dosage.

According to the present invention, volatilities of PRMS are based on absolute vapor pressure expressed in Torr. As such, top notes and middle notes are defined accordingly:

TABLE 3

| | Vp |
|---|---|
| Top | Pvap > 0.08 Torr |
| Middle | 0.0008 < Pvap < 0.08 Torr |
| Bottom | Pvap less than 0.0008 Torr |

Accordingly, in certain aspects, fixatives are selected so that:
i. They are present at dosage of 0.05% or higher, 1% or higher, or and 3% or higher in a final consumer product containing 0.5% to 95% of fragrance including the fixative molecule. The ratio of fixative to fragrance dosage may be 1:4.
ii. Solubility parameters may target top and middle notes based on the ranges described below. The fixatives have at least 2 out of 3 Hansen solubility parameters for each class defined as top and middle notes.

TABLE 4

| | Top Notes | | |
|---|---|---|---|
| | δ_{d} | δₚ | δₕ |
| Average | 15.84 | 4.15 | 6.72 |
| STDev | 3.56 | 2.65 | 4.11 |

TABLE 5

| | Middle Notes | | |
|---|---|---|---|
| | δ_{d} | δₚ | δₕ |
| Average | 16.86 | 4.61 | 7.66 |
| STDev | 2.72 | 3.10 | 3.29 |

In one aspect, the at least one fixative comprises a compound having:
i. at least two HSPs selected from a group consisting of: an atomic dispersion force (δ_{d}) from 12 to 20, a dipole moment (δₚ) from 1 to 7, and a hydrogen bonding (δₕ) from 2.5 to 11, when in solution with a compound having a vapor pressure greater than 0.08 Torr at 22° C.; and
ii. at least two HSPs selected from a second group consisting of: an atomic dispersion force (δ_{d}) from 14 to 20, a dipole moment (δₚ) from 1 to 8, and a hydrogen bonding (δₕ) from 4 to 11, when in solution with a compound having a vapor pressure range of 0.0008 to 0.08 Torr at 22° C.

In one aspect, group (i) includes at least two HSPs selected from the group consisting of: an atomic dispersion force (δ_{d}) of 15.84±3.56, a dipole moment (δₚ) of 4.15±2.65, and a hydrogen bonding (δₕ) of 6.72±4.11.

In a further aspect, group (ii) includes at least two HSPs selected from the group consisting of: an atomic dispersion force (δ_{d}) of 16.86±2.72, a dipole moment (δₚ) of 4.61±3.10, and a hydrogen bonding (δₕ) of 7.66±3.29

The at least one modulator may be, for example, selected from the compounds listed in the following table.

TABLE 6

| IUPAC | CAS# | δd | δh | δp | INCI name |
|---|---|---|---|---|---|
| octan-1-ol | 111-87-5 | 16 | 11.2 | 5 | Caprylyl Alcohol |
| octan-2-ol | 123-96-6 | 16.08 | 8.96 | 4.21 | octanol |
| 2-butyloctan-1-ol | 3913-02-8 | 16.07 | 9.2 | 3.21 | Butyloctanol |
| 11-methyldodecan-1-ol | 27458-92-0 | 15.92 | 6.64 | 3.07 | Isotridecyl Alcohol |
| 2-hexyldecan-1-ol | 2425-77-6 | 15.81 | 7.8 | 2.81 | Hexyldecanol |
| 14-methylpentadecan-1-ol | 36311-34-9 | 15.87 | 6.46 | 2.57 | Isocetyl Alcohol |
| 16-methylheptadecan-1-ol | 27458-93-1 | 15.87 | 5.9 | 2.38 | Isostearyl Alcohol |
| 2-octyldecan-1-ol | 45235-48-1 | 15.83 | 6.92 | 2.56 | Octyldecanol |
| 2-octyldodecan-1-ol | 5333-42-6 | 15.86 | 6.15 | 2.38 | Octyldodecanol |
| 2-decyltetradecan-1-ol | 58670-89-6 | 15.88 | 5.03 | 2.08 | Decyltetradecanol |
| 2-dodecylhexadecan-1-ol | 72388-18-2 | 15.87 | 4.22 | 1.86 | Dodecylhexadecanol |
| 2-tetradecyloctadecan-1-ol | 32582-32-4 | 15.85 | 3.69 | 1.71 | Tetradecyloctadecanol |
| [3-(2-ethylhexanoyloxy)-2, | 28510-23-8 | 15.91 | 6.34 | 3.6 | Neopentyl Glycol |
| 2-dimethylpropyl] | | | | | Diethylhexanoate |
| 2-ethylhexanoate | | | | | |
| 3-tetradecoxypropan-1-ol | 63793-60-2 | 16.98 | 18.27 | 9.99 | PPG-3 Myristyl Ether |

In aspects of the invention, the fixative is an aliphatic alcohol wherein the alcohol functional group is a non-terminal functional group. For example, the alcohol functional group may arise at least 2, at least 3, or at least 4 carbons from the terminal carbon. An example of such a fixative is 2-hexyldecan-1-ol:

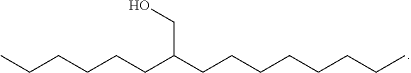 [SMILES: CCCCCCCCC(CO)CCCCCC]

An aliphatic alcohol according to the present invention may be linear or branched. The longest chain in the aliphatic alcohol is at least five carbons long. The aliphatic alcohol may be, for example, up to 32 carbons long.

Surprisingly, fixatives according to the present invention having an alcohol moiety in the middle of the carbon chain have better performance compared to a similar molecule having a terminal alcohol functional group. Known fixatives, which do not have 2 to 3 HSP values within the above ranges were found to be less effective than the fixatives of the present invention.

According to the present invention, a profragrance may be a fixative by meeting the HSP criteria. A profragrance may also be used in combination with a fixative of the present invention. The profragrance may extend freshness of the fragrance by release of its perfumery compound upon application and/or act as a fixative prior to the release of the perfumery ingredient.

### b. Perfume Ingredients

A “perfuming ingredient” or “perfume raw material” as used herein means a compound for use in perfumery, which is used for its ability to smell pleasantly and to be capable of imparting a hedonic effect, or a pleasant odor to the products into which it is incorporated, or to the surfaces, such as skin or hair, to which it is applied, on its own or in admixture with other such ingredients. A perfuming ingredient has the ability to impart or modify, in a positive or pleasant way, the odor of a composition or surface. When the latter has a malodor, the perfuming ingredient may also be capable of covering such malodor so as to render the overall perceived odor pleasant.

A “perfuming ingredient” or “perfume raw material” may encompass any suitable perfume raw material for fragrance uses, including materials such as, for example, alcohols, aldehydes, ketones, esters, ethers, acetates, nitriles, terpene hydrocarbons, nitrogenous or sulfurous heterocyclic compounds and essential oils. Naturally occurring plant and animal oils and exudates comprising complex mixtures of various chemical components are also encompassed. The individual perfume raw materials which comprise a known natural oil can be found by reference to journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994). Additionally, some perfume raw materials are supplied by the fragrance houses as mixtures in the form of proprietary specialty accords.

### EXAMPLES

### Example 1—Comparison of a Fixative Meeting 3 of the HSP Criteria with a Fixative that Meets 1 of the HSP Criteria

Jarcol I16N (hexyldecanol) (Jarchem) meets 3 of the HSP criteria of the present invention. Glucam P20 (PPG-20 methyl glucose ether) (Lubricol) meets 1 HSP parameter within the HSP ranges of this invention (see table below).

TABLE 7

| IUPAC | CAS# | δd | δh | δp | INCI name |
|---|---|---|---|---|---|
| 14-methylpenta- | 36311-34-9 | 15.87 | 6.46 | 2.57 | Isocetyl |
| decan-1-ol | 20194-48-3 | | | | Alcohol |
| (2R,3S,4S,5R,6R)- | 61849-72-7 | 16.98 | 18.27 | 9.99 | PPG-20 |
| 2-(hydroxymethyl)- | | | | | Methyl |
| 6-methoxyoxane-3, | | | | | Glucose Ether |
| 4,5-triol;2- | | | | | |
| (2-hydroxypropoxy) | | | | | |
| propan-1-ol | | | | | |

### EDT A—Reference

A reference EDT formulation (EDT A) was prepared and used as a control to evaluate fragrance performance. Water was added to ethanol. After stirring, fragrance was added to this solution. The final mixture was stirred until homogeneous.

TABLE 8
Reference EDT A

| Ingredients | Amount (% wt) | Function |
|---|---|---|
| Ethanol | 80 | Solvent |
| Water | 10 | Solvent |
| Fragrance | 10 | Fragrance |

### EDT B Containing PPG-20 Methyl Glucose Ether

PPG-20 methyl glucose ether (MGE) was mixed in ethanol and water. After stirring, fragrance was added to this mixture. The final solution was stirred until homogeneous.

TABLE 9

| Ingredients | Amount (% wt) | Function |
|---|---|---|
| Ethanol 96° | 75* | Solvent |
| Water | 10 | Solvent |
| PPG-20 methyl glucose ether | 5* | Non-odoriferous fixative |
| Fragrance | 10 | Fragrance |

MGE (PPG-20 methyl glucose ether), origin: Lubrizol.
*Testing concentrations for ICA varied as follows: 0.5%, 2%, 5% and 10% where ethanol 96° amounts were adjusted respectively as follows: 79.5%, 78%, 75% and 70%.

EDT C containing Isocetyl Alcohol

Isocetyl alcohol (ICA) was mixed in ethanol and water. After stirring, fragrance was added to this mixture. The final solution was stirred until homogeneous.

TABLE 10

| Ingredients | Amount (% wt) | Function |
|---|---|---|
| Ethanol 96° | 75* | Solvent |
| Water | 10 | Solvent |
| Isocetyl Alcohol ³⁾ | 5* | Non-odoriferous fixative |
| Fragrance | 10 | Fragrance |

1) Isocetyl Alcohol (hexadecan-1-ol), origin: Ashland.
*Testing concentrations for MGE were varied as follows: 0.5%, 2%, 5% and 10% where ethanol 96° amounts were respectively adjusted as follows: 79.5%, 78%, 75% and 70%.

Research Accord HTI (RAHTI) used in experiment 1 and 1B consisted of ingredients ranging from high volatility to low volatility ingredients at equal concentrations in Table 10A.

TABLE 10A

| IUPAC name | Vp (Torr) |
|---|---|
| (Z)-3-HEXENYL ACETATE | 1.2187 |
| ALLYL HEXANOATE | 0.6777 |
| (1RS,2RS)-2,4-dimethyl-3-cyclohexene-1- | 0.5777 |
| carbaldehyde (A) + (1RS,2SR)-2,4-dimethyl- | |
| 3-cyclohexene-1-carbaldehyde (B) | |
| methyl (1RS,2SR)-2,6,6-trimethyl-3-cyclohexene- | 0.18 |
| 1-carboxylate (A) + methyl (1RS,2RS)-2,6,6- | |
| trimethyl-3-cyclohexene-1-carboxylate (B) | |
| (2RS,4SR)-2-methyl-4-propyl-1,3-oxathiane (A) + | 0.1227 |
| (2RS,4RS)-2-methyl-4-propyl-1,3-oxathiane (B) | |
| (3Z)-3-hexen-1-yl butyrate | 0.1353 |
| METHYL BENZOATE | 0.3404 |
| (+−)-methyl 2,2-dimethyl-6- | 0.1462 |
| methylidenecyclohexanecarboxylate | |
| ETHYL BENZOATE | 0.1798 |
| 2,6-DIMETHYL-2-HEPTANOL | 0.3304 |
| (Z)-3-HEXENYL ISOBUTYRATE | 0.1817 |
| (−)-PROPYL (S)-2-(1,1- | 0.0196 |
| DIMETHYLPROPOXY)PROPANOATE | |
| (3E,5Z)-1,3,5-UNDECATRIENE | 0.3358 |
| 2,6,6-TRIMETHYL-1,3-CYCLOHEXADIENE-1- | 0.1339 |
| CARBALDEHYDE | |
| (+−)-3,7-dimethyl-1,6-octadien-3-ol | 0.0905 |
| (1R,2R)-1,7,7-TRIMETHYL- | 0.0959 |
| BICYCLO[2.2.1]HEPT-2-YL ACETATE | |
| (2RS,5SR)-5-methyl-2-(2-propanyl)cyclohexanone (A) + | 0.2559 |
| (2RS,5RS)-5-methyl-2-(2-propanyl)cyclohexanone (B) | |
| ALLYL HEPTANOATE | 0.1596 |
| (+)-(R)-3,7-DIMETHYL-6-OCTENAL | 0.2148 |
| (E)-4-DECENAL | 0.3307 |
| (3Z)-1,3-undecadien-5-yne (A) + (3E)-1,3- | 0.1998 |
| undecadien-5-yne (B) | |
| (+−)-(3Z)-3,4,5,6,6-pentamethyl-3-hepten-2-one | 0.039 |
| (A) + (+−)-3,5,6,6-tetramethyl-4-methylidene- | |
| 2-heptanone (B) + (+−)-(3E)-3,4,5,6,6- | |
| pentamethyl-3-hepten-2-one (C) + (+−)-(E)- | |
| 3,4,5,6,6-pentamethylhept-4-en-2-one (D) | |
| 1-methoxy-4-(2-propen-1-yl)benzene | 0.2102 |
| 2-PHENYLETHYL FORMATE | 0.0505 |
| (E)-3,7-DIMETHYL-1,6-NONADIEN-3-OL (A) + | 0.1307 |
| (Z)-3,7-DIMETHYL-1,6-NONADIEN-3-OL (B) | |
| (+−)-2-pentylcyclopentanone | 1.31E−01 |
| (2E)-1-[(1RS,2SR)-2,6,6-trimethyl-3- | 0.0102 |
| cyclohexen-1-yl]-2-buten-1-one | |
| 3,7-DIMETHYL-6-OCTENYL ACETATE | 0.0137 |
| (2Z)-3,7-dimethyl-2,6-octadien-1-yl acetate | 0.0256 |
| METHYL 2-NONYNOATE | 0.0568 |
| (E)-3,7-DIMETHYL-2,6-OCTADIENYL ACETATE | 0.0256 |
| ALLYL 3-CYCLOHEXYLPROPANOATE | 0.0092 |
| INDOL | 0.0298 |
| (+−)-2,4-dimethyl-4,4a,5,9b-tetrahydroindeno[1,2- | 0.00251 |
| d][1,3]dioxine (ISOMER A) + (+−)-2,4-dimethyl- | |
| 4,4a,5,9b-tetrahydroindeno[1,2-d][1,3]dioxine | |
| (ISOMER B) (A + B) | |
| (+−)-4-NONANOLIDE | 0.0086 |
| (+−)-3-(4-isopropylphenyl)-2-methylpropanal | 0.0088 |
| (+−)-4-DECANOLIDE | 0.0085 |
| 6-hexyltetrahydro-2H-pyran-2-one | 0.0019 |
| (+−)-(4E)-3-METHYL-4-CYCLOPENTADECEN- | 0.0001 |
| 1-ONE (A) + (+−)-(5E)-3-METHYL-5- | |
| CYCLOPENTADECEN-1-ONE (B) + (+−)-(5Z)- | |
| 3-METHYL-5-CYCLOPENTADECEN-1-ONE (C) | |
| (−)-(3aR,5aS,9aS,9bR)-3a,6,6,9a- | 0.0093 |
| tetramethyldodecahydronaphtho[2,1-b]furan | |
| 4-hydroxy-3-methoxybenzaldehyde | 0.0019 |
| (+)-METHYL (1R)-CIS-3-OXO-2-PENTYL-1- | 0.0007 |
| CYCLOPENTANEACETATE | |
| 2-CHROMENONE | 0.0013 |
| BENZYL 2-HYDROXYBENZOATE | 0.0002 |

Evaporations were done in Tzero lids. Prazitherm PZ72 slide warmer pre-heated to 32° C. for 30 minutes. Each crucible was placed on the precision hotplate. Using an adjustable volume pipette, 10 μL of fragrance was dosed directly to the center of the crucible and evaporated at 32° C. for 5 minutes (considered as the Time Zero), 30 minutes, 1 hour, 2 hours, 4 hours and 6 hours on the precision hotplate. A duplicate set was performed for each sample and each condition tested. When time points were reached, each crucible was placed in a 2-mL Agilent GC vial (Agilent 5183-2068) and 600 μL ethanol was added to stop the evaporation. Vials were closed and mixed by shaking for at least 1 minute. Samples were analyzed by GC-MS direct injection methodology.

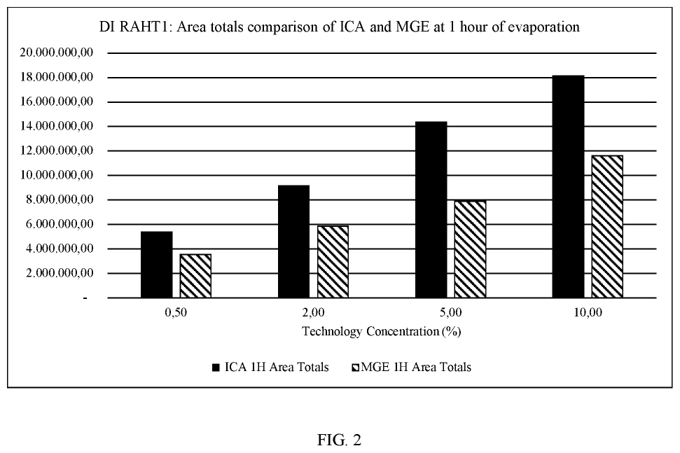

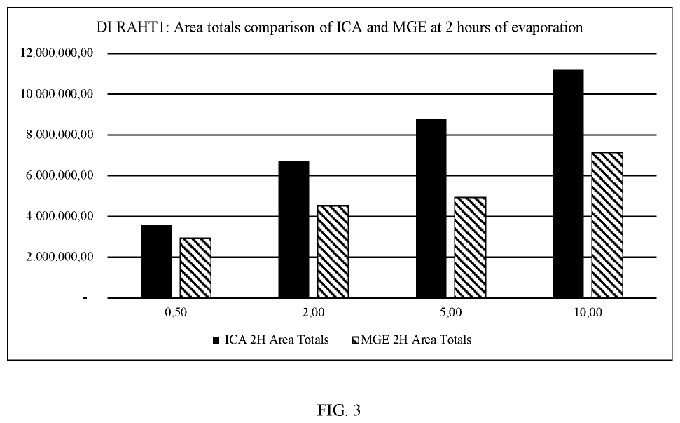

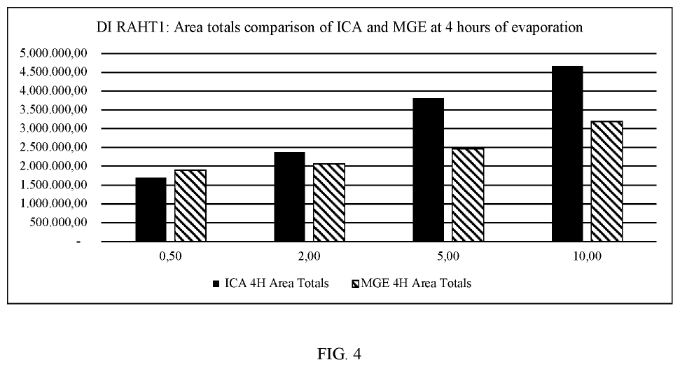

FIGS. 2 to 4 show the total area sums at 1 hour evaporation, 2 hours evaporation and 4 hours evaporation of different levels of PPG-20 Methyl Glucose Ether (EDT B) vs. different levels of Isocetyl Alcohol (EDT C).

The data shows a higher retention of the sum of all compounds with Isocetyl Alcohol (EDT C) at all levels throughout the entire evaporation against PPG-20 Methyl Glucose Ether (EDT B) at all levels except for 0.5% which shows the opposite effect at 4 hours evaporation.

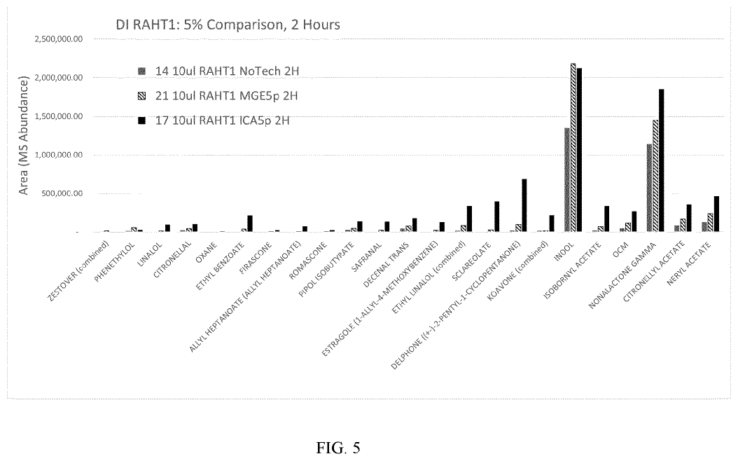

FIG. 5 shows direct injection data for each individual compound at 2 hours evaporation of RAHTI alone (EDT A) vs. 5% PPG-20 Methyl Glucose Ether with RAHTI (EDT B) vs. 5% Isocetyl Alcohol with RAHTI (EDT C).

The data shows a higher retention of all compounds with 5% Isocetyl Alcohol (EDT C) at 2 hours of evaporation against the control. The same result is observed with PPG-20 Methyl Glucose Ether (EDT B), but to a lower degree.

### Example 1B—Profragrance Meeting 2 of the HSP Criteria have fixative properties

Profragrances 1-3 meet 2 of the HSP criteria of the present invention.

| IUPAC | CAS# | δd | δh | δp | Profragrance Code |
|---|---|---|---|---|---|
| 3-(dodecylthio)-1- | 543724-31-8 | 16.51 | 1.71 | 2.15 | Profragrance 1 |
| [(1RS,2SR)-2,6,6- | | | | | |
| trimethyl-3-cyclohexen- | | | | | |
| 1-yl]-1-butanone | | | | | |
| Mixture of (+−)-4- | 1394132-28-5 | 16.52 | 1.98 | 2.69 | Profragrance 2 |
| (dodecylthio)-4-(2,6,6- | and | | | | |
| trimethyl-2-cyclohexen- | 1394132-36-5 | | | | |
| 1-yl)-2-butanone and | | | | | |
| (+−)-4-(dodecylthio)- | | | | | |
| 4-(2,6,6-trimethyl-1- | | | | | |
| cyclohexen-1-yl)-2- | | | | | |
| butanone in a ratio of | | | | | |
| 55:45 | | | | | |
| (2-((2-methylundec-1-en- | 2489743-82-8 | 16.78 | 2.76 | 2.37 | Profragrance 3 |
| 1-yl)oxy)ethyl)benzene | | | | | |

EDT D containing Profragrance 1

Profragrance 1 was mixed in ethanol and water. After stirring, fragrance was added to this mixture. The final solution was stirred until homogeneous.

| Ingredients | Amount (% wt) | Function |
|---|---|---|
| Ethanol 96° | 78 | Solvent |
| Water | 10 | Solvent |
| Profragrance 1 | 3 | Profragrance |
| Fragrance | 10 | Fragrance |

EDT E containing Profragrance 2

Profragrance 2 was mixed in ethanol and water. After stirring, fragrance was added to this mixture. The final solution was stirred until homogeneous.

| Ingredients | Amount (% wt) | Function |
|---|---|---|
| Ethanol 96° | 78 | Solvent |
| Water | 10 | Solvent |
| Profragrance 2 | 3 | Profragrance |
| Fragrance | 10 | Fragrance |

EDT F containing Profragrance 3

Profragrance 3 was mixed in ethanol and water. After stirring, fragrance was added to this mixture. The final solution was stirred until homogeneous.

| Ingredients | Amount (% wt) | Function |
|---|---|---|
| Ethanol 96° | 78 | Solvent |
| Water | 10 | Solvent |
| Profragrance 3 | 3 | Profragrance |
| Fragrance | 10 | Fragrance |

Evaporations were done in Tzero lids. Prazitherm PZ72 slide warmer pre-heated to 32° C. for 30 minutes. Each crucible was placed on the precision hotplate. Using an adjustable volume pipette, 10 μL of fragrance was dosed directly to the center of the crucible and evaporated at 32° C. for 5 minutes (considered as the Time Zero), 30 minutes, 1 hour, 2 hours, 4 hours and 6 hours on the precision hotplate. A duplicate set was performed for each sample and each condition tested. When time points were reached, each crucible was placed in a 2-mL Agilent GC vial (Agilent 5183-2068) and 600 μL ethanol was added to stop the evaporation. Vials were closed and mixed by shaking for at least 1 minute. Samples were analyzed by GC-MS direct injection methodology.

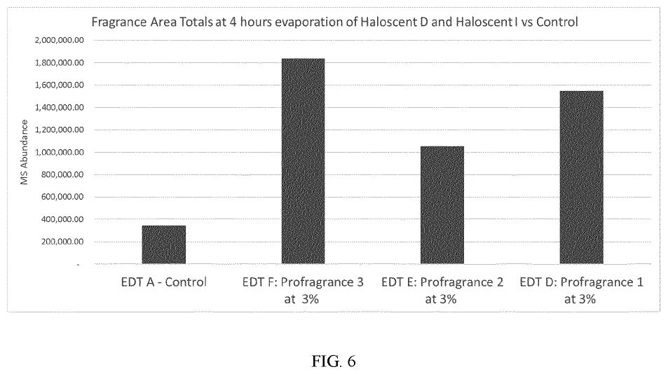

FIG. 6 shows the fragrance area sums at 4 hours evaporation of EDT D (Profragrance 1), EDT E (Profragrance 2) and EDT F (Profragrance 3) vs. control EDT A.

The data shows a higher fragrance retention at 4 hours evaporation in EDT D (Profragrance 1), EDT E (Profragrance 2) and EDT F (Profragrance 3) vs. control EDT A.

### Example 2—Fixatives with a Non-Terminal Alcohol Functional Group are efficIent as Fixatives

### EDT1—Reference

A reference EDT formulation was prepared and used as a control to evaluate fragrance performance. Water was added to ethanol. After stirring, fragrance was added to this solution. The final mixture was stirred until homogeneous.

TABLE 11

| Ingredients | Amount (% wt) | Function |
|---|---|---|
| Ethanol | 79 | Solvent |
| Water | 11 | Solvent |
| Fragrance | 10 | Fragrance |

### EDT2 Containing Hexyldecanol

Hexyldecanol was mixed in ethanol and water. After stirring, fragrance was added to this mixture. The final solution was stirred until homogeneous.

TABLE 12

| Ingredients | Amount (% wt) | Function |
|---|---|---|
| Ethanol 96° | 79 | Solvent |
| Water | 6 | Solvent |
| Hexyldecanol ¹⁾ | 5 | Non-odoriferous fixative |
| Fragrance | 10 | Fragrance |

¹⁾ Jarcol I-16N (2-hexyldecan-1-ol), origin: Jarchem Industries, Inc.

### EDT3 Containing Octyldodecanol

Octyldodecanol was mixed in ethanol and water. After stirring, fragrance was added to this mixture. The final solution was stirred until homogeneous.

TABLE 13

| Ingredients | Amount (% wt) | Function |
|---|---|---|
| Ethanol 96° | 79 | Solvent |
| Water | 6 | Solvent |
| Octyldodecanol ¹⁾ | 5 | Non-odoriferous fixative |
| Fragrance | 10 | Fragrance |

¹⁾ Jarcol I-20N (2-Octyl-1-dodecanol), origin: Jarchem Industries, Inc.

### Evaporation GC-MS Kinetics

The following evaporation kinetic studies were performed. Research Accord HT2 (RAHT2) used in this experiment consisted of ingredients ranging from high volatility to low volatility ingredients at equal concentrations.

TABLE 14

| IUPAC | Vp (Torr) |
|---|---|
| ETHYL BUTANOATE | 13.941 |
| BUTYL ACETATE | 11.545 |
| 3-METHYLBUTYL ACETATE (A) + | 7.8529 |
| (+−)-2-METHYLBUTYL ACETATE (B) | |
| (2E)-2-HEXENAL | 4.6238 |
| 3-METHYL-2-BUTENYL ACETATE | 3.9867 |
| (+−)-ETHYL 2-METHYLPENTANOATE | 2.9056 |
| beta-PINENE 89% (A) + alpha-PINENE 11% (B) | 3.4891 |
| ETHYL HEXANOATE | 1.6649 |
| BENZALDEHYDE | 0.9742 |
| 1,3,3-trimethyl-2-oxabicyclo[2.2.2]octane | 1.6485 |
| (Z)-3-HEXEN-1-OL | 1.0394 |
| TETRAHYDRO-4-METHYL-2-(2-METHYL-1- | 0.5513 |
| PROPENYL)-2H-PYRAN | |
| (+−)-2,6-DIMETHYL-5-HEPTENAL | 0.622 |
| 3-METHYLBUTYL BUTANOATE (A) + 2- | 0.8898 |
| METHYLBUTYL BUTANOATE (B) | |
| HEXYL ACETATE | 1.3908 |
| (3Z)-hex-3-en-1-yl methyl carbonate | 0.7206 |
| 6,6-DIMETHOXY-2,5,5-TRIMETHYL-2-HEXENE | 0.2144 |
| nonanal | 0.5321 |
| (2E,6Z)-2,6-NONADIENAL | 0.2801 |
| benzyl acetate | 0.1637 |
| (+−)-2,6-DIMETHYL-7-OCTEN-2-OL | 0.1656 |
| (2,2-DIMETHOXYETHYL)BENZENE | 0.5561 |
| (2RS,5SR,9RS,10SR)-2,6,9,10-tetramethyl-1- | 0.0213 |
| oxaspiro[4.5]deca-3,6-diene (A) | |
| (+−)-2,2,5-trimethyl-5-pentylcyclopentanone | 0.0261 |
| ETHYL PHENYLACETATE | |
| (+−)-1,5-DIMETHYL-1-VINYL-4-HEXENYL | 0.1158 |
| ACETATE | |
| (−)-(R)-3,7-DIMETHYL-6-OCTENENITRILE | 0.0247 |
| (3Z)-3-hexen-1-yl (3Z)-3-hexenoate | 0.0122 |
| 2-PHENYLETHYL ACETATE | 0.0564 |
| 1-methoxy-4-[(1E)-1-propen-1-yl]benzene | 0.0687 |
| (+−)-(E)-3-METHYL-4-(2,6,6-TRIMETHYL-2- | |
| CYCLOHEXEN-1-YL)-3-BUTEN-2-ONE | |
| (+−)-(2E)-1-(2,6,6-trimethyl-2- | 0.0083 |
| cyclohexen-1-yl)-2-buten-1-one | |
| 3-(4-ETHYLPHENYL)-2,2-DIMETHYLPROPANAL (A) + | 0.0081 |
| 3-(2-ETHYLPHENYL)-2,2-DIMETHYLPROPANAL (B) | |
| ALLYL (CYCLOHEXYLOXY)ACETATE | 0.0032 |
| (+-)-1-(2-TERT-BUTYL-1-CYCLOHEXYLOXY)-2- | 0.000803 |
| BUTANOL | |
| (+-)-7-HYDROXY-3,7-DIMETHYLOCTANAL | 0.0032 |
| (+-)-3-methyl-5-(2,2,3-trimethyl-3- | 0.0006 |
| cyclopenten-1-yl)-2-pentanol | |
| (+)-(1S,1′R)-2-[1-(3′,3′-DIMETHYL-1′- | 0.0001 |
| CYCLOHEXYL)ETHOXY]-2-METHYLPROPYL | |
| PROPANOATE | |
| (ETHOXYMETHOXY)CYCLODODECANE | 0.0043 |
| (+−)-3-(1,3-BENZODIOXOL- | 0.0027 |
| 5-YL)-2-METHYLPROPANAL | |
| (+−)-5-heptyldihydro-2(3H)-furanone | 0.0027 |
| 9-ACETYL-8-CEDRENE + | |
| CEDARWOOD SESQUITERPENES | |
| 1,4-dioxacycloheptadecane-5,17-dione | 0 |

RAHT2 was solubilized in EDT1, EDT2 and EDT3.

Evaporations were done in Tzero lids. Prazitherm PZ72 slide warmer pre-heated to 32° C. for 30 minutes. Each crucible was placed on the precision hotplate. Using an adjustable volume pipette, 10 μL of fragrance was dosed directly to the center of the crucible and evaporated at 32° C. for 5 minutes (considered as the Time Zero), 30 minutes, 1 hour, 2 hours, 4 hours and 6 hours on the precision hotplate. A duplicate set was performed for each sample and each condition tested. When time points were reached, each crucible was placed in a 2-mL Agilent GC vial (Agilent 5183-2068) and 600 μL ethanol was added to stop the evaporation. Vials were closed and mixed by shaking for at least 1 minute. Samples were analyzed by GC-MS direct injection methodology.

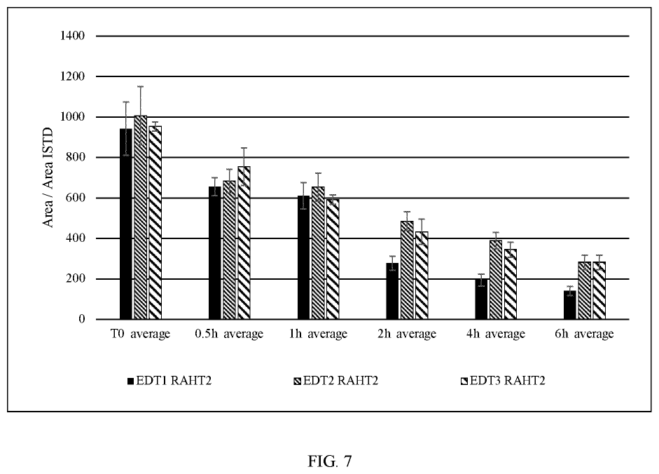

FIG. 7 shows the total area sums throughout evaporation of RAHT2 alone (EDT1 RAHT2) vs. 5% Hexyldecanol with RAHT2 (EDT2 RAHT2) vs. 5% Octyldodecanol with RAHT2 (EDT3 RAHT2).

The data shows a higher retention of the sum of all compounds with 5% Hexyldecanol (EDT2) throughout the entire evaporation against the control. The same result was observed with 5% Octyldodecanol (EDT3).

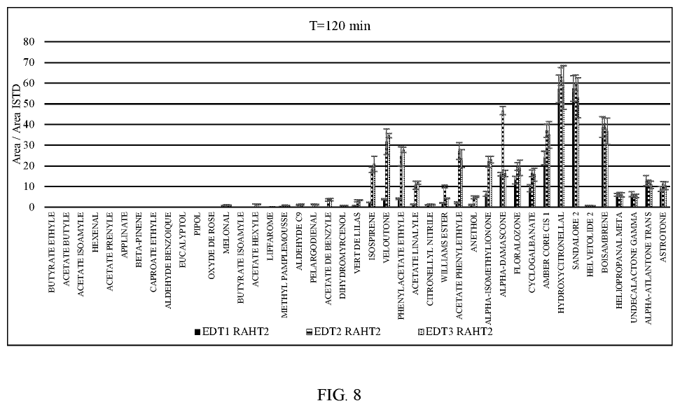

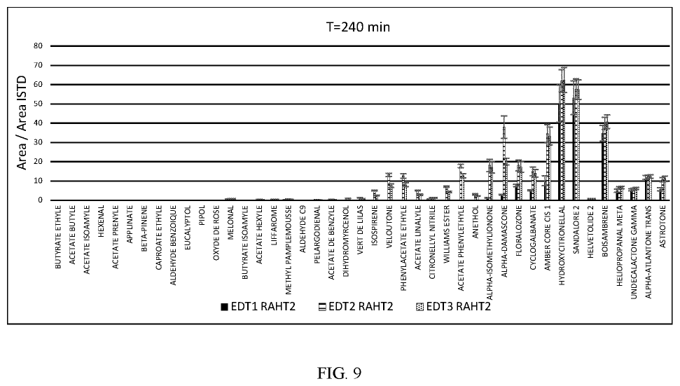

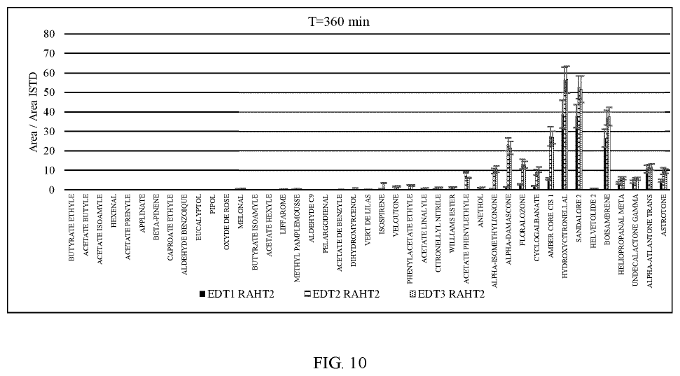

FIGS. 8-10 show direct injection data for each individual compound at 2 hours evaporation, 4 hours evaporation and 6 hours evaporation of RAHT2 alone (EDT1 RAHT2) vs. 5% Hexyldecanol with RAHT2 (EDT2 RAHT2) vs. 5% Octyldodecanol with RAHT2 (EDT3 RAHT2).

The data shows a higher retention of all compounds, most noticeably mid and low volatility notes.

### Sensory Evaluation

A sensory evaluation of overall intensity was performed on fragrance CF which consists of the ingredients below.

TABLE 15

| IUPAC | Parts (wt %) | Vp(Torr) |
|---|---|---|
| decanal | 0.1099 | 0.2073 |
| OCTANAL | 0.1099 | 2.0679 |
| 1-[(2RS,3RS,8aRS)-2,3,8,8-tetramethyl- | 5.4945 | 0.0006 |
| 1,2,3,5,6,7,8,8a-octahydro-2- | | |
| naphthalenyl]ethanone | | |
| benzyl acetate | 0.3297 | 0.1637 |
| (−)-(5R)-5-isopropenyl-2-methyl-2- | 0.1099 | 0.0656 |
| cyclohexen-1-one | | |
| (2Z)-3,7-dimethyl-2,6-octadienal | 3.2967 | 0.0712 |
| (+−)-3,7-DIMETHYL-6-OCTEN-1-OL | 0.1099 | 0.0183 |
| (+−)-3-methyl-5-(2,2,3-trimethyl-3- | 1.6484 | 0.0006 |
| cyclopenten-1-yl)-2-pentanol | | |
| ethyl (2E,4Z)-2,4-decadienoate | 0.5495 | 0.0095 |
| (3E,5Z)-1,3,5-UNDECATRIENE* | 0.1099 | 0.3358 |
| LAVENDER OIL | 0.6593 | — |
| 1-Methyl-4-(prop-1-en-2-yl)cyclohex-1-ene | 25.2747 | — |
| (+−)-4-isopropeny1-1-methylcyclohexene | 21.9780 | 1.541 |
| (−)-(3R)-3,7-DIMETHYL-1,6- | 7.9121 | 0.0905 |
| OCTADIEN-3-OL | | |
| (+−)-3,7-dimethyl-1,6-octadien-3-yl | 21.9780 | 0.0392 |
| acetate | | |
| methyl 2-(methylamino)benzoate | 0.3297 | 0.0193 |
| PETITGRAIN OIL | 4.3956 | — |
| [2-[1-(3,3-dimethylcyclohexyl)ethoxy]- | 5.4945 | 0.000124 |
| 2-oxoethyl] propanoate | | |
| (1RS,2SR,4RS)-2-methoxy-4- | 0.1099 | 0.004 |
| propylcyclohexanol | | |

*supplied at 10% in triethyl citrate

Prazitherm PZ72 slide warmer was pre-heated to 32° C. for 30 minutes. Glass plates were placed on the precision hotplate. Using an adjustable volume pipette, 20 μl of EDT was dosed directly to the center of the glass plate and evaporated at 32° C. At different times (t =0 min (Fresh), 2 hours, 4 hours and 6 hours), the randomized glass plates were evaluated by 7 panelists.

A 3-Alternative Forced Choice (3-AFC) test was used. For each time point, panelists were presented with 3 samples, two of which were the fragrance CF (EDT1), and one was the fragrance CF according to invention (EDT2 or EDT3). the present Panelists indicated the sample(s) that they perceived higher in terms of overall intensity.

### Hypothesis:

H0: The two samples are not different.
H1: The sample with technology is more intense than the sample without technology, in terms of overall intensity.

### Associated Risks:

H0 rejected=αa risk:
Risk associated with a false alarm, concluding that products differ when in fact they do not.

Data was analyzed using the binomial statistic.

### Data Interpretation:

If the p-value obtained for α≤0.05, then the sample with technology was more intense in overall intensity than the sample without technology,
If the p-value obtained for α is 0.05<α>0.10, then a trend difference was determined,
If the p-value obtained for α>0.10, the samples were not significantly different.

The results of the sensory panel presented in the table below show a higher performance of the formulation according to the present invention at fresh and after 4 hours of evaporation in the presence of 5% Hexyldecanol (EDT2), and at all time points in the presence of 5% Octyldodecanol (EDT3).

TABLE 16

| Samples | Nb of | p-value | | | |
|---|---|---|---|---|---|
| tested | panelists | Fresh | 2 h | 4 h | 6 h |
| EDT1 CF vs. | 7 | 0.0005 | 0.7366 | 0.0069 | 0.0005 |
| EDT2 CF | | | | | |
| EDT1 CF vs. | 7 | 0.0005 | 0.0069 | 0.0069 | 0.0069 |
| EDT3 CF | | | | | |

### Example 3: Hexyldecanol has Better Long-Lasting Benefit in Sensory than ICA

### EDT2 Containing Hexyldecanol

Hexyldecanol was mixed in ethanol and water. After stirring, fragrance was added to this mixture. The final solution was stirred until homogeneous.

TABLE 17

| Ingredients | Amount (% wt) | Function |
|---|---|---|
| Ethanol 96° | 79 | Solvent |
| Water | 6 | Solvent |
| Hexyldecanol ¹⁾ | 5 | Non-odoriferous fixative |
| Fragrance | 10 | Fragrance |

¹⁾ Jarcol I-16N (2-hexyldecan-1-ol), origin: Jarchem Industries, Inc.

### EDT4 Containing Isocetyl Alcohol

Isocetyl alcohol was mixed in ethanol and water. After stirring, fragrance was added to this mixture. The final solution was stirred until homogeneous.

TABLE 18

| Ingredients | Amount (% wt) | Function |
|---|---|---|
| Ethanol 96° | 79 | Solvent |
| Water | 6 | Solvent |
| Isocetyl alcohol ¹⁾ | 5 | Non-odoriferous fixative |
| Fragrance | 10 | Fragrance |

¹⁾ Ceraphyl ICA (14-methylpentadecan-1-ol), origin: Ashland

### Sensory Evaluation

A sensory evaluation of overall intensity was performed on fragrance CF.

Prazitherm PZ72 slide warmer was pre-heated to 32° C. for 30 minutes. Glass plates were placed on the precision hotplate. Using an adjustable volume pipette, 20 μl of EDT was dosed directly to the center of the glass plate and evaporated at 32° C. At different times (t=0 min (Fresh), 2 hours, 4 hours and 6 hours), the randomized glass plates were evaluated by 7 panelists.

A 3-Alternative Forced Choice (3-AFC) test was used. For each time point, panelists were presented with 3 samples, two of which were the fragrance CF (EDT 3), and one was the fragrance CF according to the present invention (EDT2). Panelists indicated the sample(s) that they perceived higher in terms of overall intensity.

### Hypothesis:

H0: The two samples are not different.
H1: The sample with technology is more intense than the sample without technology, in terms of overall intensity.

### Associated Risks:

H0 rejected=αrisk:
Risk associated with a false alarm, concluding that products differ when in fact they do not.

Data was analyzed using the binomial statistic.

### Data Interpretation:

If the p-value obtained for α≤0.05, then the sample with technology was more intense in overall intensity than the sample without technology,
If the p-value obtained for α is 0.05<α<0.10, then a trend difference was determined,
If the p-value obtained for α>0.10, the samples were not significantly different.

The results of the sensory panel data below show a higher performance of the formulation according to the present invention after 2H and after 4 hours of evaporation in the presence of 5% Hexyldecanol (EDT2).

TABLE 19

| Samples | Nb of | p-value | | | | |
|---|---|---|---|---|---|---|
| tested | panelists | Fresh | 30 min. | 1 h | 2 h | 4 h |
| EDT4 CF vs. | 7 | 0.7366 | 0.9415 | 0.1733 | 0.0453 | 0.0453 |
| EDT2 CF | | | | | | |

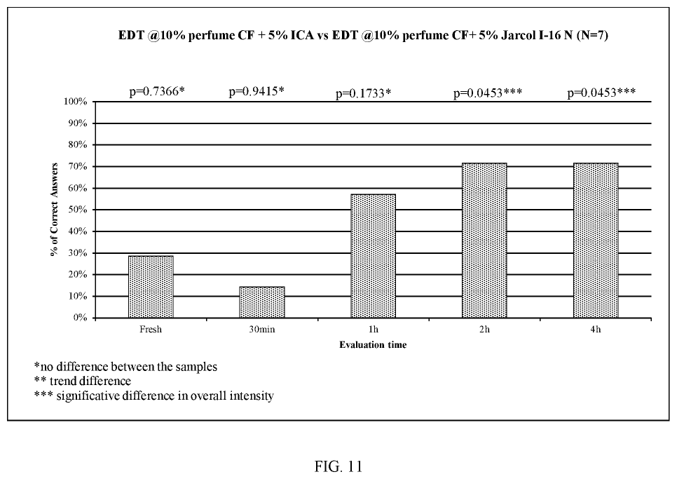

FIG. 11 shows that a fragrance with hexylcecanol was perceived more intense after 2 hours and 4 hours dry down compared to fragrance with ICA.

1. A fragrance comprising:
(a) at least one perfume ingredient selected from the group consisting of: a perfume ingredient having a top note, a perfume ingredient having a middle note, and a combination thereof; and
(b) at least one fixative having:
i. at least two Hansen Solubility Parameters selected from a first group consisting of: an atomic dispersion force (δ_{d}) from 12 to 20, a dipole moment (δₚ) from 1 to 7, and a hydrogen bonding (δₕ) from 2.5 to 11, when in solution with a compound having a vapor pressure greater than 0.08 Torr at 22° C.; and
ii. at least two Hansen Solubility Parameters selected from a second group consisting of: an atomic dispersion force (δ_{d}) from 14 to 20, a dipole moment (δₚ) from 1 to 8, and a hydrogen bonding (δₕ) from 4 to 11, when in solution with a compound having a vapor pressure range of 0.0008 to 0.08 Torr at 22° C.

2. The fragrance of claim 1, wherein group (i) is selected from the group consisting of: an atomic dispersion force (δ_{d}) of 15.84±3.56, a dipole moment (δₚ) of 4.15±2.65, and a hydrogen bonding (δₕ) of 6.72±4.11; and group (ii) is selected from the group consisting of: an atomic dispersion force (δ_{d}) of 16.86±2.72, a dipole moment (δₚ) of 4.61±3.10, and a hydrogen bonding (δₕ) of 7.66±3.29.

3. The fragrance of claim 1, wherein the at least one fixative is selected from the group consisting of: octan-1-ol, octan-2-ol, 2-butyloctan-1-ol, 11-methyldodecan-1-ol, 2-hexyldecan-1-ol, 14-methylpentadecan-1-ol, 16-methylheptadecan-1-ol, 2-octyldecan-1-ol, 2-octyldodecan-1-ol, 2-decyltetradecan-1-ol, 2-dodecylhexadecan-1-ol, 2-tetradecyloctadecan-1-ol, [3-(2-ethylhexanoyloxy)-2,2-dimethylpropyl] 2-ethylhexanoate, 3-tetradecoxypropan-1-ol, and a combination thereof.

4. The fragrance of claim 1, wherein the fixative is non-odiferous.

5. The fragrance of claim 1, further comprising a volatile solvent and water.

6. The fragrance of claim 1, wherein the fixative comprises 0.1% or greater by weight of the fragrance.

7. The fragrance of claim 1, wherein the fixative is in a ratio of at least 1/10 to the at least one perfume ingredient.

8. The fragrance of claim 7, wherein the fixative is in a ratio of at least 1/4 compared to the at least one perfume ingredient.

9. The fragrance of claim 1, wherein the fixative is represented by the formula (I):
CH₃-(CH₂)ₓ-CHZ-(CH₂)_{y}-(CH₃) (I)
wherein Z is a CH₂OH, a CHO, a CO₂H, a CH₂NH₂ or a CH₂SH group;
x is an integer between 3 and 15;
y is an integer between 3 and 15; and
provided that |x-y| is less than 8.

10. The fragrance of claim 1, wherein the fixative is a profragrance.

11. A consumer product comprising the fragrance of claim 1.
